(19) United States
(12) Patent Application Publication
(10) Pub. No.: US 2024/0081683 A1
(43) Pub. Date: Mar. 14, 2024

(54) BALLISTOCARDIOGRAM SENSOR

(71) Applicant: Electronics and Telecommunications Research Institute, Daejeon (KR)

(72) Inventors: Chaehyun LIM, Daejeon (KR); Hye Jin KIM, Daejeon (KR)

(73) Assignee: Electronics and Telecommunications Research Institute, Daejeon (KR)

(21) Appl. No.: 18/364,081

(22) Filed: Aug. 2, 2023

(30) Foreign Application Priority Data
Sep. 13, 2022 (KR) 10-2022-0114860

(51) Int. Cl. A61B 5/11

(52) U.S. Cl. CPC A61B 5/1102

(57) ABSTRACT

A ballistocardiogram sensor according to an embodiment of the inventive concept includes a lower substrate including a support part, a plurality of connection parts, and a vertical movement part, a lower electrode on the lower substrate, a piezoelectric sensing layer on the lower electrode, and an upper electrode on the piezoelectric sensing layer. The vertical movement part is spaced apart from the support part with the connection parts therebetween. The connection parts connect the vertical movement part and the support part. The piezoelectric sensing layer vertically overlaps the vertical movement part. The connection parts may have a serpentine shape.

### CROSS-REFERENCE TO RELATED APPLICATIONS

This U.S. non-provisional patent application claims priority under 35 U.S.C. § 119 of Korean Patent Application No. 10-2022-0114860, filed on Sep. 13, 2022, the entire contents of which are hereby incorporated by reference.

### BACKGROUND

The present disclosure herein relates to a technology pertaining to a ballistocardiogram sensor.

Ballistocardiogram (BCG) represents signals or waveforms obtained by measuring vibration (ballistic) due to changes in blood flow in the heart and blood vessels in response to the contraction and relaxation of the heart, and is an index that indicates cardiac activity similarly to electrocardiogram and can be usefully used in health monitoring in everyday life.

A ballistocardiogram sensor, which is a device for measuring vibration generated due to changes in blood flow in the heart and blood vessels in response to the contraction and relaxation of the heart, is used to predict or diagnose functional abnormalities of the heart such as abnormal heart beat and abnormal blood flow. In general, the ballistocardiogram sensor includes a piezo film, and may perform measurement by sensing a change in voltage that occurs due to physical deformation of the piezo film caused by vibration.

### SUMMARY

The present disclosure provides a structure of a patch-type ballistocardiogram sensor capable of amplifying weak physical vibration of a heart.

An embodiment of the inventive concept provides a ballistocardiogram sensor including: a lower substrate including a support part, a plurality of connection parts, and a vertical movement part; a lower electrode on the lower substrate; a piezoelectric sensing layer on the lower electrode; and an upper electrode on the piezoelectric sensing layer, wherein the vertical movement part is spaced apart from the support part with the connection parts therebetween, the connection parts connect the vertical movement part and the support part, the piezoelectric sensing layer vertically overlaps the vertical movement part, and the connection parts have a serpentine shape.

In an embodiment, the lower substrate may include a polyimide.

In an embodiment, the lower electrode may include: a first contact part on the support part; a first connection pattern on the connection part; and a capping pattern on the vertical movement part, wherein the contact part and the capping pattern may be spaced apart from each other and connected to the first connection pattern, and the first connection pattern may vertically overlap the connection part and have a serpentine shape.

In an embodiment, the upper electrode may include: a second contact part on the support part; a second connection pattern on the connection part; and a crossing pattern on the vertical movement part, wherein the second connection pattern may vertically overlap the connection part and have a serpentine shape.

In an embodiment, the capping pattern and the crossing pattern may contact the piezoelectric sensing layer.

In an embodiment, the first connection pattern and the second connection pattern may be spaced apart from each other, and the first contact part and the second contact part may contact each other.

In an embodiment, the crossing pattern may have a comb shape.

In an embodiment, the upper electrode may be provided in plurality, the crossing pattern of one of the upper electrodes may include a first horizontal structure extending in a first direction and first vertical structures extending from the first horizontal structure in a second direction intersecting the first direction, and the crossing pattern of other one of the upper electrodes may include a second horizontal structure spaced apart from the first horizontal structure in the second direction and extending in the first direction and second vertical structures extending in the second direction, wherein the first vertical structures and the second vertical structures may be alternately and repeatedly arranged.

In an embodiment, the upper electrode may be provided in plurality, the crossing patterns of one pair of the upper electrodes may be locally arranged in a first region of the vertical movement part, and the crossing patterns of other one pair of the upper electrodes may be locally arranged in a second region of the vertical movement part.

In an embodiment, the vertical movement part may include thereon a plurality of bump patterns formed integrally.

In an embodiment, a height of the bump patterns may increase in a direction from a center portion of the vertical movement part to an outer periphery of the vertical movement part.

In an embodiment, a spacing distance between the bump patterns may increase in a direction from a center portion of the vertical movement part to an outer periphery of the vertical movement part.

In an embodiment, the bump patterns may be arranged in a matrix, spiral, or radial form.

In an embodiment, the vertical movement part may have one of polygon shapes in a plan view, and the connection parts may be respectively connected to vertices of the polygon.

In an embodiment, the piezoelectric sensing layer may include a PVDF-TrFE material.

### BRIEF DESCRIPTION OF THE FIGURES

The accompanying drawings are included to provide a further understanding of the inventive concept, and are incorporated in and constitute a part of this specification. The drawings illustrate embodiments of the inventive concept and, together with the description, serve to explain principles of the inventive concept. In the drawings:

FIG. 1 is a perspective view of a lower structure of a ballistocardiogram sensor;

FIG. 2 is a perspective view of an upper structure of a ballistocardiogram sensor;

FIG. 3 is an exploded perspective view of a ballistocardiogram sensor;

FIG. 4 is a perspective view of a ballistocardiogram sensor;

FIGS. 5A to 5F are plan views illustrating a lower substrate of a ballistocardiogram sensor;

FIGS. 6A and 6B are plan views schematically illustrating a ballistocardiogram sensor;

FIGS. 7A and 7B are cross-sectional views taken along line I-I′ of FIG. 1;

FIGS. 8A to 8C are plan views illustrating a bump pattern of a ballistocardiogram sensor;

FIG. 9 is a graph illustrating signal processing for a separated signal after a signal separation process for removing noise from a measured ballistocardiogram signal;

FIG. 10 is a graph illustrating a ballistocardiogram signal subjected to signal processing according to an embodiment; and

FIG. 11 is a graph illustrating a typical ballistocardiogram signal according to a comparative example.

### DETAILED DESCRIPTION

Hereinafter, embodiments of the inventive concept will be described with reference to the accompanying drawings so that the configuration and effects of the inventive concept are sufficiently understood. However, the inventive concept is not limited to the embodiments described below, but may be implemented in various forms and may allow various modifications. Rather, these embodiments are provided so that this disclosure will be thorough and complete, and will fully convey the scope of the inventive concept to those skilled in the art. In the accompanying drawings, the dimensions of elements are magnified for convenience, and the scale ratios among the elements may be exaggerated or reduced.

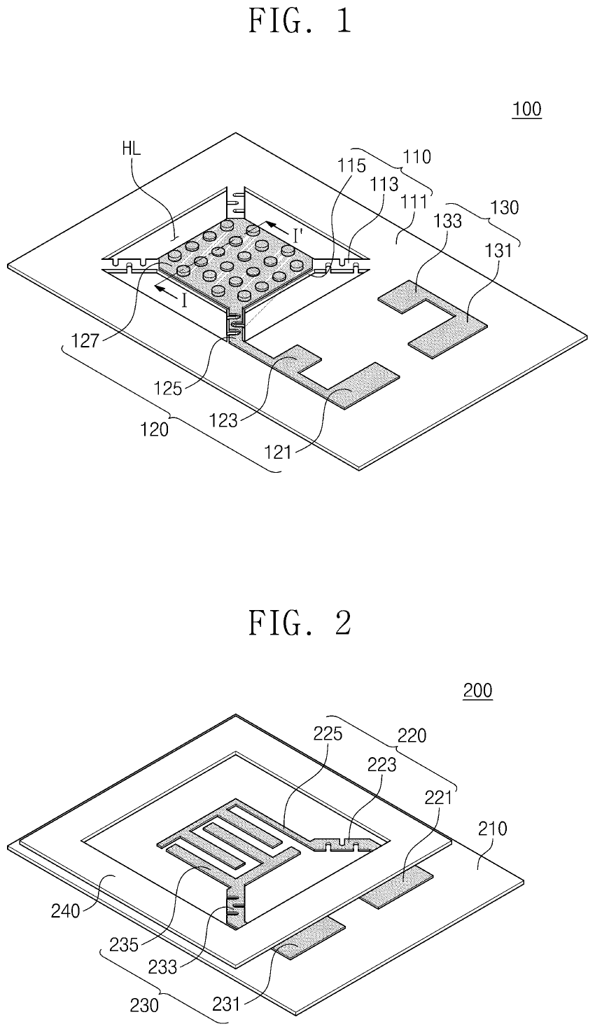

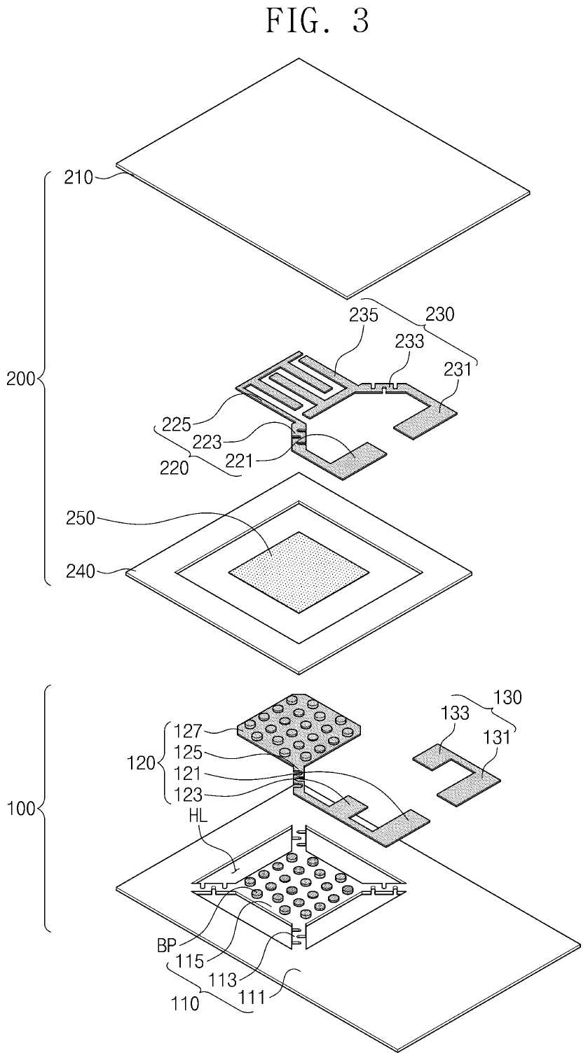

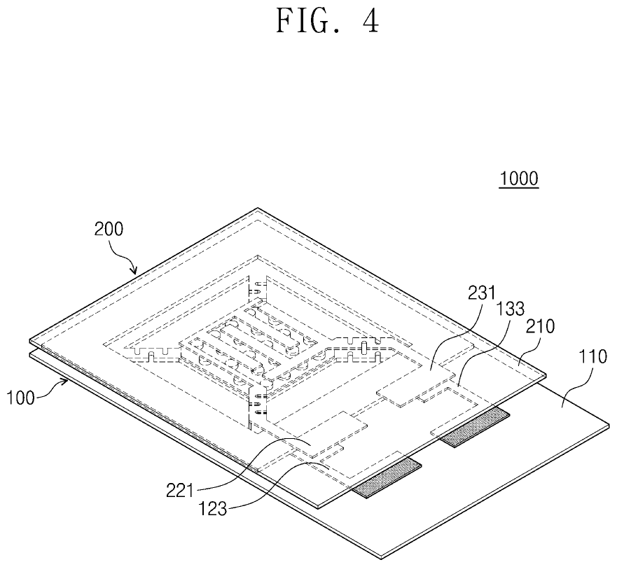

FIG. 1 is a perspective view of a lower structure of a ballistocardiogram sensor. FIG. 2 is a perspective view of an upper structure of a ballistocardiogram sensor. FIG. 3 is an exploded perspective view of a ballistocardiogram sensor. FIG. 4 is a perspective view of a ballistocardiogram sensor.

Referring to FIGS. 1, 2, and 3, a ballistocardiogram sensor 1000 may include a lower structure 100 and an upper structure 200.

Referring to FIGS. 1 and 3, the lower structure 100 may include a lower substrate 110, a first lower electrode 120, and a second lower electrode 130. The lower substrate 110 has a shape of a sheet and may include a flexible polymer material. For example, the lower substrate 110 may include a polyimide. The lower substrate 110 may include a support part 111, a connection part 113, and a vertical movement part 115 that are integrated.

The support part 111 may occupy a largest area of the lower substrate 110, and may be a part attached to a human body.

The connection part 113 may have a shape of a beam structure. Herein, the connection part 113 may be referred to as a beam structure. The beam structure may be provided in plurality, and each of beam structures may have a flexible shape such as a serpentine shape, repetitive S shape, grooved zigzag shape, spiral shape, etc.

The vertical movement part 115 may have a shape of a plate, and a planar shape thereof may include various shapes such as a quadrangle, a circle, etc. Herein, the vertical movement part 115 may be referred to as a membrane structure. The vertical movement part 115 may be spaced apart from the support part 111 with the connection part 113 therebetween. The support part 111 and the vertical movement part 115 may be spaced apart from each other by an opening HL, and the connection parts 113 adjacent to each other may also be spaced apart from each other by the opening HL therebetween. Bump patterns BP may be provided on the vertical movement part 115. The bump patterns BP may include a plurality of cylinders or square pillars arranged in a form of zigzag, M×N matrix (both M and N are natural numbers larger than 2), or the like. The bump patterns BP each may have a shape protruding from an upper surface of the vertical movement part 115. The bump patterns BP may include the same material as the vertical movement part 115 and may be integrally connected thereto. The bump patterns BP may amplify vibration/pressure applied to a piezoelectric sensing layer 250 that will be described later, and may maximize frictional electricity characteristics of the piezoelectric sensing layer 250.

When the vertical movement part 115 receives minute external force due to vibration of a human chest, the connection parts 113 may concentrate the external force so as to allow the vertical movement part 115 to move with a large displacement in a direction perpendicular to the external force. As a result, the vertical movement part 115 may more effectively deliver, to the piezoelectric sensing layer 250, vibration/pressure delivered in a vertical direction. Furthermore, a shape of the connection part 113 arranged in a serpentine form and in a form of a plurality of spirals may minimize horizontal movement (left-right movement) of the vertical movement part 115. For example, considering that systolic/diastolic blood pressure of blood vessels generated due to contraction and relaxation of a heart is typically 120/80 mmHg, pressure/vibration delivered to the outside of a thoracic cavity may be amplified to an amplitude of about 400 μm even under pressure of about 0.003% of the systolic/diastolic blood pressure according to an embodiment of the inventive concept.

A first lower electrode 120 and a second lower electrode 130 may be arranged on the lower substrate 110. The first lower electrode 120 and the second lower electrode 130 may be spaced apart from each other in a direction parallel with the upper surface of the lower substrate 110. The first lower electrode 120 and the second lower electrode 130 may include a metal material, for example, gold (Au).

The first lower electrode 120 may include a first lower contact part 121, a second lower contact part 123, a lower connection pattern 125, and a capping pattern 127. The first lower contact part 121, the second lower contact part 123, the lower connection pattern 125, and the capping pattern 127 may be integrally connected.

The first lower contact part 121 may be arranged on the support part 111 of the lower substrate 110. The first lower contact part 121, for example, may be a portion to which ground voltage is applied.

The second lower contact part 123 may be arranged on the support part 111 of the lower substrate 110. The second lower contact part 123 may be a portion that contacts or is electrically connected to a first upper electrode 220 that will be described later. The second lower contact part 123 may be arranged between the first lower contact part 121 and the lower connection pattern 125. The lower connection pattern 125 may be arranged on the connection part 113 of the lower substrate 110. The lower connection pattern 125 may have a shape that is the same as or similar to a pattern of the connection part 113. The capping pattern 127 may cover the upper surface of the vertical movement part 115 and an upper surface and side surface of the bump patterns BP.

The second lower electrode 130 may include a third lower contact part 131 and a fourth lower contact part 133. Unlike the first lower electrode 120, the second lower electrode 130 may not include a connection pattern and a capping pattern. The third lower contact part 131 may be a portion through which a signal is output. The fourth lower contact part 133 may be a portion that contacts or is electrically connected to a second upper electrode 230 that will be described later.

The bump patterns BP may contact the piezoelectric sensing layer 250 through the capping pattern 127. The bump patterns BP may reduce an area of contact with the piezoelectric sensing layer 250 so that heart vibration in a chest may be locally concentrated on the piezoelectric sensing layer 250. As a result, an effect of at least about 80% pressure amplification is brought about in comparison with the case where the capping pattern 127 having a planar shape without the bump patterns BP directly contacts the piezoelectric sensing layer 250.

Referring to FIGS. 2 and 3, the upper structure 200 may include an upper substrate 210, the first upper electrode 220, the second upper electrode 230, a spacer 240, and the piezoelectric sensing layer 250. FIG. 2 illustrates a rotated state in which the upper substrate 210 is located at a lowermost position, and does not illustrate the piezoelectric sensing layer 250 of FIG. 3 for clearer illustration.

The upper substrate 210 has a shape of a sheet and may include a flexible polymer material. For example, the upper substrate 210 may include a polyimide.

The first upper electrode 220 and the second upper electrode 230 may be arranged on the upper substrate 210. The first upper electrode 220 and the second upper electrode 230 may be arranged spaced apart from each other. The first upper electrode 220 and the second upper electrode 230 may include a metal material, for example, gold.

The first upper electrode 220 may include a first upper contact part 221, a first upper connection pattern 223, and a first crossing pattern 225.

The first upper contact part 221 may directly contact or may be electrically connected to the first lower contact part 121 of the first lower electrode 120 and may receive ground voltage.

The first upper contact part 221 and the first crossing pattern 225 may be spaced apart from each other, and the first upper connection pattern 223 may connect the first upper contact part 221 and the first crossing pattern 225. The first upper connection pattern 223 may have a shape that is the same as or similar to a pattern of the connection part 113.

The first crossing pattern 225 may include a first horizontal structure extending in a first direction and a plurality of first vertical structures extending in a second direction intersecting the first direction like a shape of a comb.

The second upper electrode 230 may have a configuration that is substantially the same as or similar to the first upper electrode 220. The second upper electrode 230 may include a second upper contact part 231, a second upper connection pattern 233, and a second crossing pattern 235.

The second upper contact part 231 may directly contact the fourth lower contact part 133 of the second lower electrode 130 or may output a signal thereto.

The second upper contact part 231 and the second crossing pattern 235 may be spaced apart from each other, and the second upper connection pattern 233 may connect the second upper contact part 231 and the second crossing pattern 235. The second upper connection pattern 233 may have a shape that is the same as or similar to a pattern of the connection part 113. The second crossing pattern 235 may include a second horizontal structure extending in a first direction and a plurality of second vertical structures extending in a second direction intersecting the first direction like a shape of a comb.

The piezoelectric sensing layer 250 may be provided on the firsts upper electrode 220 and the second upper electrode 230. The piezoelectric sensing layer 250 may be a nano fiber-type film. The piezoelectric sensing layer 250 may include a polymer material such as PVDF-TrFE material.

The spacer 240 may be provided on the upper substrate 210. The spacer 240 may prevent the first lower electrode 120 and the first upper electrode 220 from contacting each other except for contact parts thereof, and may prevent the second lower electrode 130 and the second upper electrode 230 from contacting each other except for contact parts thereof. The spacer 240 may have a shape of a frame including an opening.

Referring to FIGS. 3 and 4, the upper structure 100 and the lower structure 200 may be coupled to each other to form the ballistocardiogram sensor 1000.

The piezoelectric sensing layer 250 may be arranged between the first lower electrode 120 and the first upper electrode 220 and second upper electrode 230. In detail, the piezoelectric sensing layer 250 may contact the capping pattern 127 of the first lower electrode 120, the first crossing pattern 225 of the first upper electrode 220, and the second crossing pattern 235 of the second upper electrode 230. The upper structure 100 and the lower structure 200 may be coupled to each other so that parts other than the first contact part 121 of the first lower electrode 120 and the third contact part 131 of the second lower electrode 220 may not be exposed to the outside.

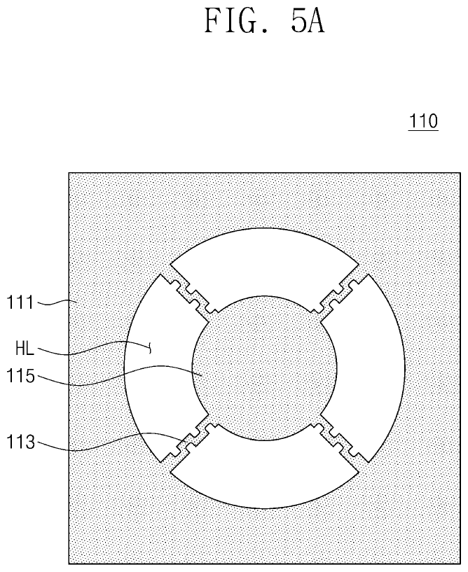

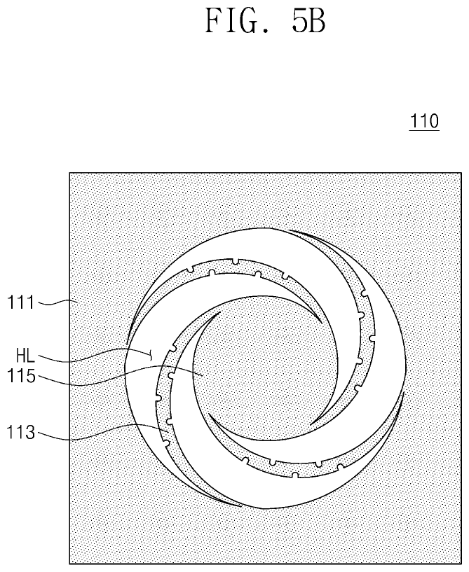

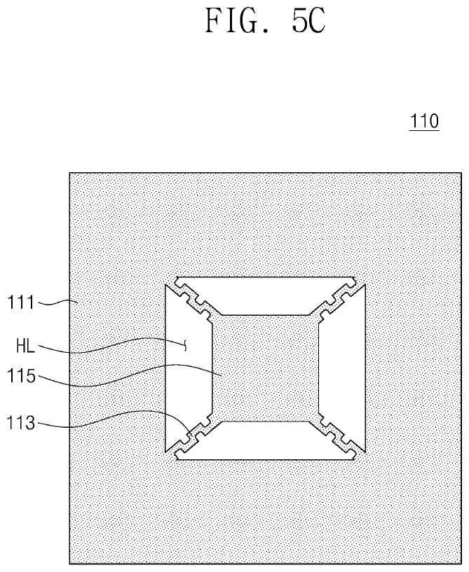

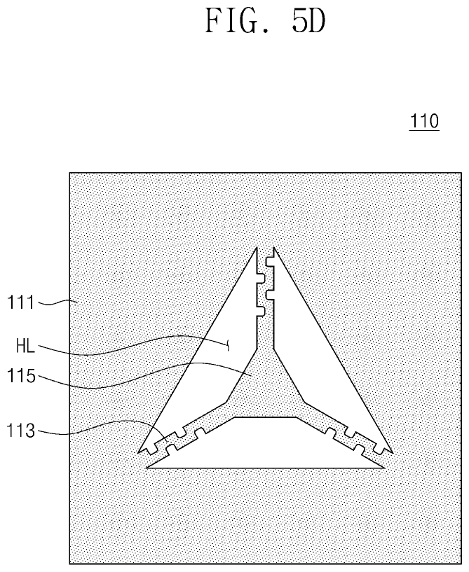

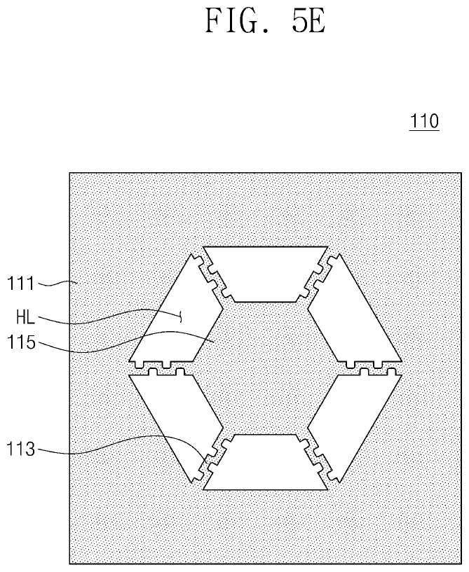

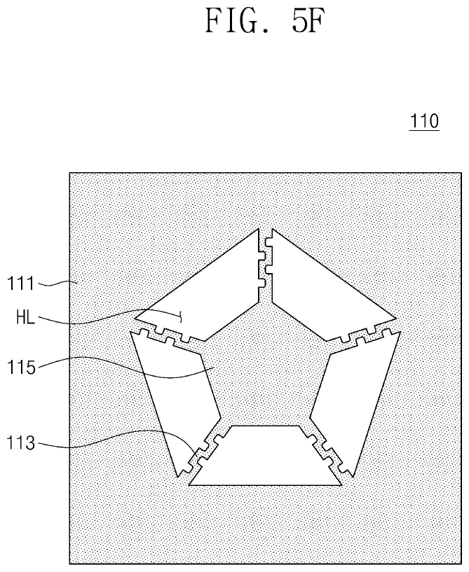

FIGS. 5A to 5F are plan views illustrating a lower substrate of a ballistocardiogram sensor.

Referring to FIG. 5A, the vertical movement part 115 may be circular or near circular in a plan view. The openings HL may have a shape of a segmented round ring surrounding the vertical movement part 115. Four of the connection parts 113 may be spaced apart from each other so as to surround the vertical movement part 115. The connection parts 113 may be grouped as two pairs that are arranged symmetrically with the vertical movement part 115 therebetween.

Normal stress applied to the connection parts 113 may be proportional to a length of the connection parts 113. An anchor position between the vertical movement part 115 and the connection parts 113 for fixing the vertical movement part 115 is designed to maintain equal angles between intersection lines of the connection parts 113 so as to uniformly distribute mechanical stress.

Referring to FIG. 5B, the vertical movement part 115 may be circular or near circular in a plan view. The openings HL and the connection parts 113 may have a shape of a spiral surrounding the vertical movement part 115.

Referring to FIG. 5C, the vertical movement part 115 may be rectangular or near rectangular in a plan view. The openings HL may have a shape of a segmented rectangular ring surrounding the vertical movement part 115. The openings HL each may have a near trapezoidal shape. Four of the connection parts 113 may be arranged spaced apart from each other at positions near vertices of the vertical movement part 115 so as to surround the vertical movement part 115. The connection parts 113 may be grouped as two pairs that are arranged symmetrically with the vertical movement part 115 therebetween. The vertical movement part 115 may have a polygonal shape in a plan view, and the connection parts 113 may be respectively connected to vertices of a polygon.

Referring to FIG. 5D, the vertical movement part 115 may be near triangular. The openings HL may have a shape of a segmented triangular ring surrounding the vertical movement part 115. The openings HL each may have a near trapezoidal shape. Three of the connection parts 113 may be arranged spaced apart from each other at positions near vertices of the vertical movement part 115 so as to surround the vertical movement part 115.

Referring to FIG. 5E, the vertical movement part 115 may be near hexagonal. The openings HL may have a shape of a segmented hexagonal ring surrounding the vertical movement part 115. The openings HL each may have a near trapezoidal shape. Six of the connection parts 113 may be arranged spaced apart from each other at positions near vertices of the vertical movement part 115 so as to surround the vertical movement part 115.

Referring to FIG. 5F, the vertical movement part 115 may be near pentagonal. The openings HL may have a shape of a segmented pentagonal ring surrounding the vertical movement part 115. The openings HL each may have a near trapezoidal shape. Five of the connection parts 113 may be arranged spaced apart from each other at positions near vertices of the vertical movement part 115 so as to surround the vertical movement part 115.

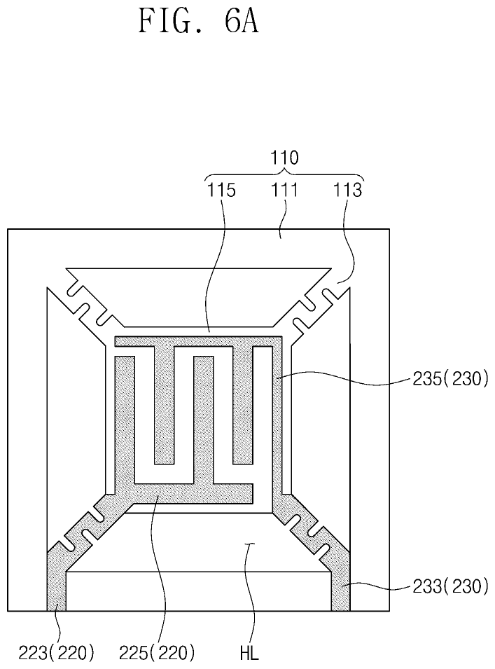

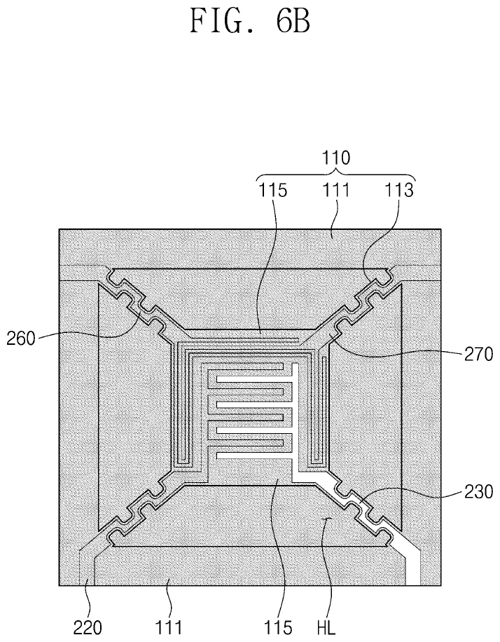

FIGS. 6A and 6B are plan views schematically illustrating a ballistocardiogram sensor. Components other than a lower substrate and an upper electrode are not illustrated.

Referring to FIG. 6A, the first upper connection pattern 223 of the first upper electrode 220 and the second upper connection pattern 233 of the second upper electrode 230 may have a flexible shape such as a serpentine shape, repetitive S shape, grooved zigzag shape, spiral shape, etc. like the connection pater 113 of the lower substrate 110. The first upper connection pattern 223 and the second upper connection pattern 233 may vertically overlap the connection part 113.

The first crossing pattern 225 of the first upper electrode 220 and the second crossing pattern 235 of the second upper electrode 230 may intersect each other. For example, the first horizontal structure of the first crossing pattern 225 may be spaced apart from the second horizontal structure of the second crossing pattern 235 in a first direction. Furthermore, the first vertical structures of the first crossing pattern 225 may be arranged in a second direction and alternately with the second vertical structures of the second crossing pattern 235.

Furthermore, as illustrated in FIG. 6B, upper electrodes 260 and 270 may be further added and arranged on an outer periphery of the vertical movement part 115, and may be differentiated from signals of the upper electrodes 220 and 230 on a center portion of the vertical movement part 115.

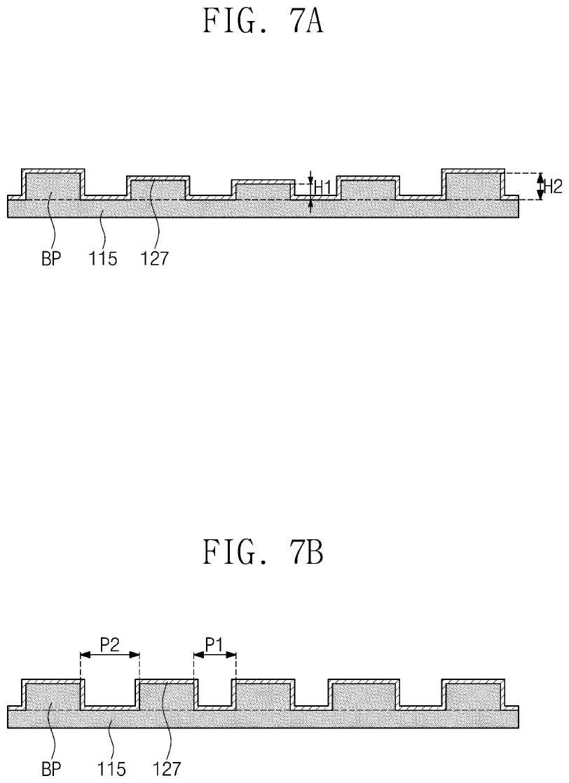

FIGS. 7A and 7B are cross-sectional views taken along line I-I′ of FIG. 1.

Referring to FIG. 7A, the bump patterns BP may have a first height H1 at a center portion of the vertical movement part 115 and a second height H2, which is greater than the first height H1, at an outer periphery of the vertical movement part 115. The height of the bump patterns BP may increase in a direction from the center portion to the outer periphery of the vertical movement part 115. In this case, the bump patterns BP may be differentiated so that the bump patterns BP on the outer periphery of the vertical movement part 115 may first react to movement due to human body activity, and the bump patterns BP on the center portion may react only to a ballistocardiogram signal. When a signal is generated by the bump patterns BP on the outer periphery but a signal is not generated on the bump patterns BP on the center portion, the signal may be identified as a signal due to human body movement rather than a ballistocardiogram signal. That is, a ballistocardiogram signal and a motion artifact signal may be more efficiently distinguished, and the motion artifact signal may be removed through a signal processing algorithm so as to improve accuracy of a ballistocardiogram sensor.

Referring to FIG. 7B, the bump patterns BP may be arranged according to a first spacing distance P1 at the center portion of the vertical movement part 115, and may be arranged according to a second spacing distance P2, which is greater than the first spacing distance P1, at the outer periphery of the vertical movement part 115. The spacing distance between the bump patterns BP or the pitch thereof may increase in a direction from the center portion to the outer periphery of the vertical movement part 115. Considering that the center portion of the vertical movement part 115 has a largest vertical displacement, stress generated due to contact between the bump patterns BP and the piezoelectric sensing layer 250 may be reduced, and uniform and stable biometric signals may be obtained.

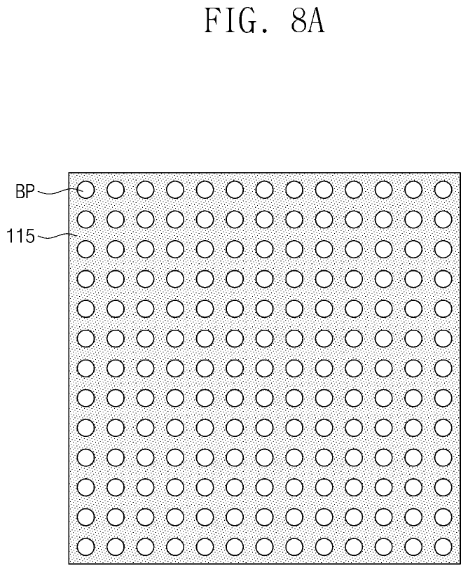

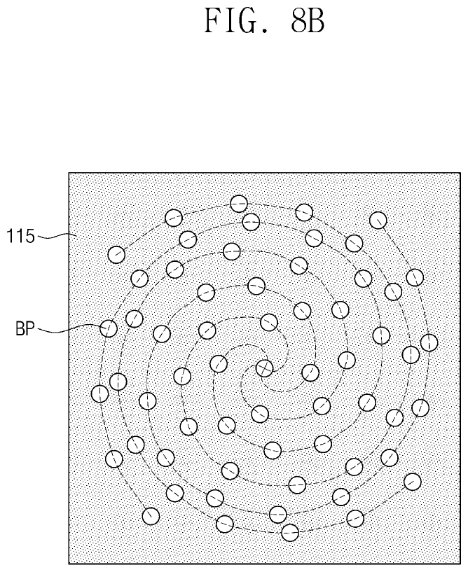

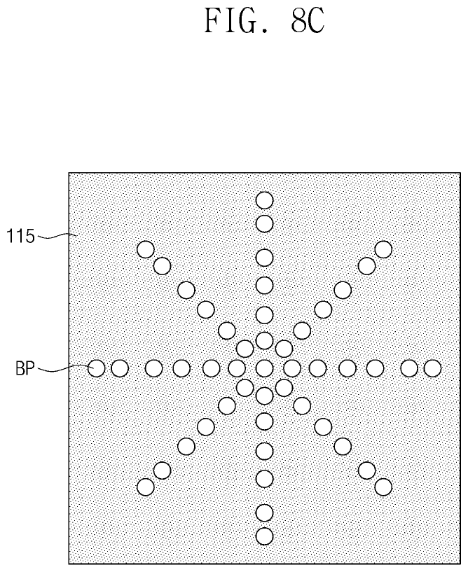

FIGS. 8A to 8C are plan views illustrating a bump pattern of a ballistocardiogram sensor.

Referring to FIG. 8A, the bump patterns BP may be arranged at a fixed pitch in a matrix form.

Referring to FIG. 8B, the bump patterns BP may be arranged in a form of a plurality of spirals with one bump pattern BP at a center.

Referring to FIG. 8C, the bump patterns BP may be arranged in a radial form.

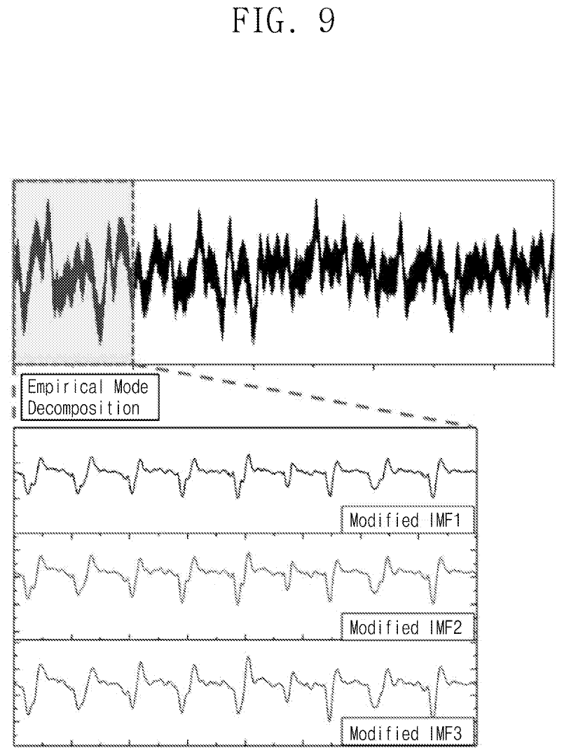

FIG. 9 is a graph illustrating signal processing for a separated signal after a signal separation process for removing noise from a measured ballistocardiogram signal. In detail, a signal obtained through an embodiment is processed by calculating intrinsic mode functions IMF1, IMF2, and IMF3 using an empirical mode decomposition method.

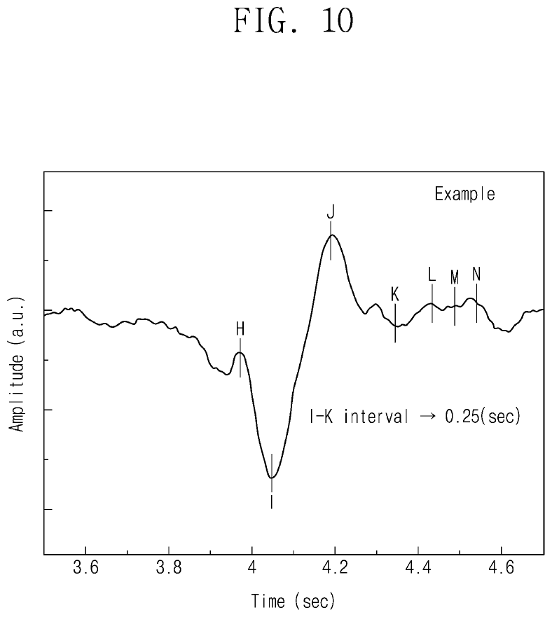

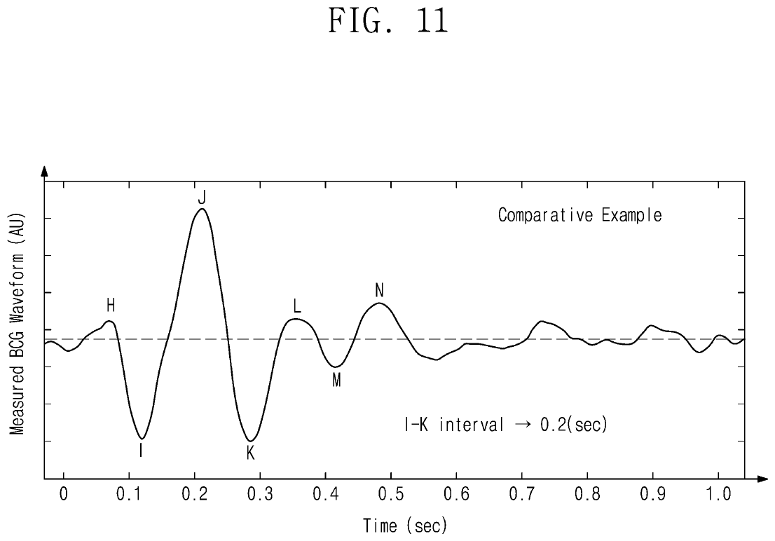

FIG. 10 is a graph illustrating a ballistocardiogram signal subjected to signal processing according to an embodiment. FIG. 11 is a graph illustrating a typical ballistocardiogram signal according to a comparative example.

Comparing FIG. 10 with FIG. 11, it may be understood that peaks H, I, J, and K and signals from K to M are also measured in the ballistocardiogram signal graph according to an embodiment similarly to the comparative example.

Typical ballistocardiogram sensors are limited in that accurate biometric information measurement is possible only when a stable posture of a patient is secured since the typical ballistocardiogram sensors are vulnerable to movement of a target for measurement or surrounding noise and thus measurement error occurs due to a thickness of clothes or a material of a sheet in which the sensors are mounted. Furthermore, constancy of measurement decreases since real-time monitoring is difficult due to movement of a patient, and collected information has low reliability and requires multiple corrections.

According to an embodiment of the inventive concept, a ballistocardiogram sensor may be attached to human skin, and ballistocardiogram may be continuously monitored in a nonrestraint manner.

In addition, the ballistocardiogram sensor according to an embodiment of the inventive concept is a patch-type sensor and is not limited in addition of electrocardiogram electrodes, and enables convergence design and modularization of the ballistocardiogram sensor and electrocardiogram electrodes. That is, electrocardiogram and ballistocardiogram may be simultaneously measured by forming an additional electrode layer on a skin attachment surface of the lower substrate 110 of an embodiment of the inventive concept. A ballistocardiogram/electrocardiogram complex sensor patch according to an embodiment of the inventive concept may be used to detect a pre-ejection period (PEP) when the heart's aortic valve opens by analyzing an R-J interval of peak R of an electrocardiogram (ECG) signal and peak J of a ballistocardiogram signal that are action potential characteristics of the heart. The PEP may be identified on the basis of the peak R that is an electric signal when the aortic valve opens and the peak J of a ballistocardiogram signal that is a maximum value of a physical signal when blood flows after the aortic valve opens, and a pulse arrival time (PAT) at which blood arrives at extremities of a human body (extremities of human limbs) may be estimated from the PEP. Since the PAT is an index for estimating characteristics and blood pressure of arteries, it may be possible to provide blood pressure information based on analysis of correlation between signals by using the ballistocardiogram/electrocardiogram complex sensor patch.

The ballistocardiogram sensor of an embodiment of the inventive concept may be attached to a user's chest and may sensitively detect minute vibration and pressure changes of heart beat that repeatedly occur from the chest, and, in this manner, may calculate ballistocardiogram that is one of pieces of physical bioactivity information of the heart.

In particular, the ballistocardiogram sensor may sensitively measure a minute ballistocardiogram signal from a chest through a vertical movement structure designed/manufactured to efficiently deliver and amplify minute heart beat vibration and a bump pattern that induces pressure amplification by reducing an area of contact with a piezoelectric sensing layer.

Although the embodiments of the present invention have been described, it is understood that the present invention should not be limited to these embodiments but various changes and modifications can be made by one ordinary skilled in the art within the spirit and scope of the present invention as hereinafter claimed.

What is claimed is:

1. A ballistocardiogram sensor comprising:
a lower substrate including a support part, a plurality of connection parts, and a vertical movement part;
a lower electrode on the lower substrate;
a piezoelectric sensing layer on the lower electrode; and
an upper electrode on the piezoelectric sensing layer,
wherein the vertical movement part is spaced apart from the support part with the connection parts therebetween,
the connection parts connect the vertical movement part and the support part,
the piezoelectric sensing layer vertically overlaps the vertical movement part, and
the connection parts have a serpentine shape.

2. The ballistocardiogram sensor of claim 1, wherein the lower substrate includes a polyimide.

3. The ballistocardiogram sensor of claim 1, wherein the lower electrode includes:
a first contact part on the support part;
a first connection pattern on the connection part; and
a capping pattern on the vertical movement part,
wherein the contact part and the capping pattern are spaced apart from each other and connected to the first connection pattern, and
the first connection pattern vertically overlaps the connection part and has a serpentine shape.

4. The ballistocardiogram sensor of claim 3, wherein the upper electrode includes:
a second contact part on the support part;
a second connection pattern on the connection part; and
a crossing pattern on the vertical movement part,
wherein the second connection pattern vertically overlaps the connection part and has a serpentine shape.

5. The ballistocardiogram sensor of claim 4, wherein the capping pattern and the crossing pattern contact the piezoelectric sensing layer.

6. The ballistocardiogram sensor of claim 4, wherein the first connection pattern and the second connection pattern are spaced apart from each other, and the first contact part and the second contact part contact each other.

7. The ballistocardiogram sensor of claim 4, wherein the crossing pattern has a comb shape.

8. The ballistocardiogram sensor of claim 4,
wherein the upper electrode is provided in plurality,
the crossing pattern of one of the upper electrodes includes a first horizontal structure extending in a first direction and first vertical structures extending from the first horizontal structure in a second direction intersecting the first direction, and
the crossing pattern of other one of the upper electrodes includes a second horizontal structure spaced apart from the first horizontal structure in the second direction and extending in the first direction and second vertical structures extending in the second direction,
wherein the first vertical structures and the second vertical structures are alternately and repeatedly arranged.

9. The ballistocardiogram sensor of claim 4,
wherein the upper electrode is provided in plurality,
the crossing patterns of one pair of the upper electrodes are locally arranged in a first region of the vertical movement part, and
the crossing patterns of other one pair of the upper electrodes are locally arranged in a second region of the vertical movement part.

10. The ballistocardiogram sensor of claim 1, wherein the vertical movement part includes thereon a plurality of bump patterns formed integrally.

11. The ballistocardiogram sensor of claim 10, wherein a height of the bump patterns increases in a direction from a center portion of the vertical movement part to an outer periphery of the vertical movement part.

12. The ballistocardiogram sensor of claim 10, wherein a spacing distance between the bump patterns increases in a direction from a center portion of the vertical movement part to an outer periphery of the vertical movement part.

13. The ballistocardiogram sensor of claim 10, wherein the bump patterns are arranged in a matrix, spiral, or radial form.

14. The ballistocardiogram sensor of claim 1, wherein the vertical movement part has one of polygon shapes in a plan view, and the connection parts are respectively connected to vertices of the polygon.

15. The ballistocardiogram sensor of claim 1, wherein the piezoelectric sensing layer includes a PVDF-TrFE material.
